(19) United States
(12) Patent Application Publication
(10) Pub. No.: US 2002/0177582 A1
(43) Pub. Date: Nov. 28, 2002

(54) Vaginal applicator

(75) Inventor: Cynthia M. Maloney, Albany, NY (US)

Correspondence Address: HESLIN ROTHENBERG FARLEY & MESITI PC, 5 COLUMBIA CIRCLE, ALBANY, NY, 12203, US

(73) Assignee: SETON HEALTH, Troy, NY

(21) Appl. No.: 10/155,515

(22) Filed: May 23, 2002

Related U.S. Application Data
(60) Provisional application No. 60/293,358, filed on May 24, 2001.

(51) Int. Cl. A61K 31/56; A61K 49/00; A61M 5/315

(52) U.S. Cl. 514/182; 424/9.1; 604/236

(57) ABSTRACT

A method for diagnosing and treating urogenital disorders associated with atrophic vaginitis in symptomatic women having vaginal pH of 4.5 or above allows documentation of symptoms, justifies estrogen use and can be used to determine the duration of treatment. The method includes measuring and recording vaginal pH, and intravaginally administering a dose of estrogen using a prefilled applicator. The applicator comprises an elongated curved tubular reservoir for containing a gel, cream or ointment, and a plunger, sealingly seatable within the reservoir and operable to displace estrogen gel, cream or ointment therefrom. The outside diameter of the reservoir ranges from 0.25 to 0.375 inches.

### CROSS-REFERENCE TO RELATED APPLICATIONS

This application claims priority from U.S. Provisional Application Serial No. 60/293,358, filed on May 24, 2001.

### FIELD OF THE INVENTION

The invention relates to methods for documenting, monitoring and treating urogenital disorders associated with atrophic vaginitis in symptomatic women having vaginal pH of 4.5 or above.

### BACKGROUND

Urinary tract infections are prevalent among residents of nursing homes and long-term care facilities, and are a major cause of morbidity for these women. (Ouslander, et al., J. Am. Geriatr. Soc., 44: 420-423 (1996)) One cause or at least one aggravator of urinary incontinence is atrophic vaginitis, which can result from the decline in estrogens during and following menopause.

Diagnosing and treating atorphic vaginitis as a transient cause of urinary incontinence is recommended by the United States Agency for Health Care Policy and Research Clinical Practice Guidelines. Local estrogen is indicated for the treatment of atrophic vaginitis, and several forms of estrogen for vaginal administration are available, including creams, vaginal ring and pills. Methods of delivering the treatment tend to be inconvenient, uncomfortable and/or unsanitary, particularly when used in a nursing home setting for women suffering from the dryness, pain and tenderness resulting from atrophic vaginitis. In addition, no standard methodology for diagnosing atrophy is commonly used. Rather, various indicators including physical symptoms such as petechiae, patient reports of vaginal dryness and laboratory reports from pap smears are used various practitioners in various situations. Because of this inconsistency, there is a need for a consistent, inexpensive, and accurate way of diagnosing atrophy.

In particular, in nursing homes and long-term care facilities, there exists a need for an easy and effective way of diagnosing and documenting atrophy and justifying estrogen use, because of the higher level of oversight and regulation. There exists a further need for a convenient, comfortable and sanitary method for delivering local estrogen for women diagnosed with vaginal atrophy.

### SUMMARY

A novel method for diagnosing and treating urogenital disorders associated with atrophic vaginitis in symptomatic women having vaginal pH of 4.5 or above, using a standard, simple, easy to use protocol, has been developed to meet the needs of patients and providers in nursing homes and long-term care facilities. The protocol allows documentation of symptoms, justifies estrogen use and can be used to determine the duration of treatment.

The method includes measuring and recording vaginal pH, and intravaginally administering a dose of estrogen using a prefilled applicator. In this context, the term ‘estrogen’ includes natural, synthetic, and semi-synthetic compounds, including estradiol, diethyl stilbestrol, estrone, estrone sodium sulfate, sodium equilin sulfate, ethinyl estradiol, quinestrol, diethylstilbestriol, mestranol, estriol, and chlorotrianisene. The applicator comprises a single dose of the estrogen composition, an elongated curved or angled tubular reservoir for containing a gel, cream or ointment, and a plunger, sealingly seatable within the reservoir and operable to displace estrogen gel, cream or ointment therefrom. The outside diameter of the reservoir ranges from 0.25 to 0.375 inches.

In another aspect, the invention relates to a kit for treatment of urogenital disorders associated with atrophic vaginitis in symptomatic women having vaginal pH of 4.5 or above. The kit includes a plurality of single doses of an estrogen composition, in the form of a gel, cream or ointment, a plurality of prefilled, disposable vaginal applicators, and instructions for administering the estrogen composition intravaginally using the applicator.

In yet another aspect, the invention relates to a prefilled vaginal applicator for intravaginal administeration of an estrogen composition in the form of a gel, cream or ointment.

In still another aspect, the invention relates to a combination device for measuring vaginal pH and for intravaginally administering a pharmaceutical composition in the form of a gel, cream or ointment. The combination device includes an elongated curved or angled tubular reservoir for containing the gel, cream or ointment, said reservoir having an outside diameter ranging from 0.25 to 0.375 inches; a plunger, sealingly seatable within the reservoir and operable to displace the gel, cream or ointment therefrom; and means for measuring vaginal pH, disposed on an external surface of the applicator. The means for measuring pH is attached to a removable protective cap, and at least a portion of an external surface of the removable protective cap, for contact with the vaginal wall, may be lubricious.

Finally, the invention also relates to a method for treating urogenital disorders associated with atrophic vaginitis in symptomatic women having vaginal pH of 4.5 or above. The method includes intravaginally administering at least one dose of an estrogen composition, in the form of a gel, cream or ointment, using a prefilled applicator comprising a single dose of the estrogen composition, an elongated curved or angled tubular reservoir containing the single dose, said reservoir having an outside diameter ranging from 0.25 to 0.375 inches, and a plunger, sealingly seatable within the reservoir and operable to displace the estrogen composition therefrom.

### BRIEF DESCRIPTION OF THE DRAWINGS

FIG. 1 is a side view of a curved applicator according to the present invention.

FIG. 2 is a side view of an applicator having an angled tip.

FIG. 3 is a side view of an applicator having a removable cap.

### DETAILED DESCRIPTION OF THE INVENTION

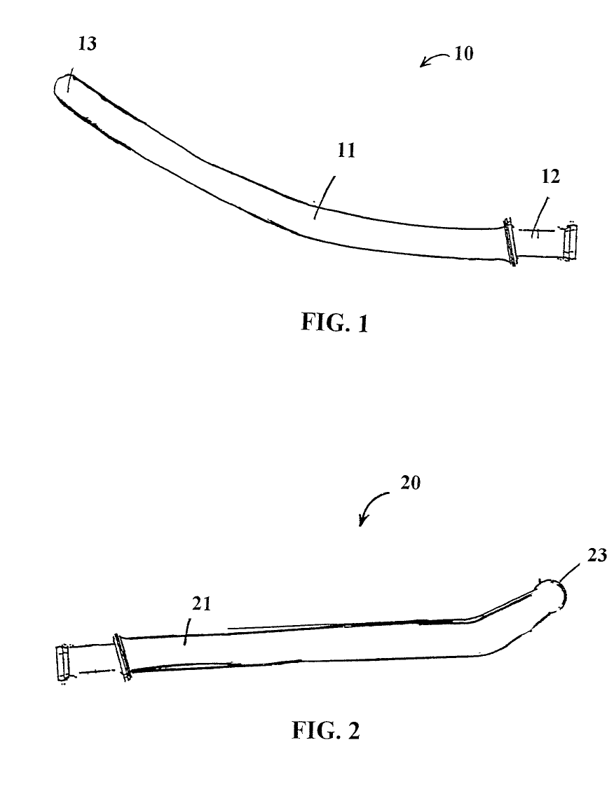

In one embodiment, the present invention relates to a method for documenting, monitoring and treating urogenital disorders associated with atrophic vaginitis in symptomatic women having vaginal pH of 4.5 or above. The method comprises measuring and recording vaginal pH, and intravaginally administering a dose of estrogen, in the form of a gel, cream or ointment, using a prefilled disposable applicator. FIGS. 1 and 2 show two embodiments of an applicator according to the present invention. In FIG. 1, applicator 10 includes an elongated curved tubular reservoir 11 for containing the estrogen a gel, cream or ointment. Reservoir 11 is typically circular or oval in cross-section, and is smaller in diameter than many vaginal applicators, having an outside diameter ranging from 0.25 to 0.375 inches (about 6 to 10 mm). Reservoir 11 may be sized to contain a 0.5-1 g dose of estrogen cream. Tip 13 of reservoir 11 is rounded and has an opening (not shown) for delivery of the contents. Since the atrophic vagina is typically tender and narrow, the applicator is fabricated from a flexible soft plastic, such as high density polyethylene or medical grade PVC. The plastic is soft for maximum comfort, but is firm enough to maintain integrity of the applicator when it is inserted into the vagina. The applicator may additionally have a lubricious external surface for contact with the vaginal wall; the lubricious area may be limited to a part of the external surface, such as tip 13. Lubricity may be provided by known biocompatible materials, for example, silicone oils or fluoropolymers. Alternately, a water-soluble lubricant may be used before insertion of the applicator. Applicator 10 also includes a piston or plunger 12, which is sealingly seatable within the reservoir and operable to displace the gel, cream or ointment therefrom. FIG. 2 shows an applicator 20 having an angled tip 23 terminating reservoir 21.

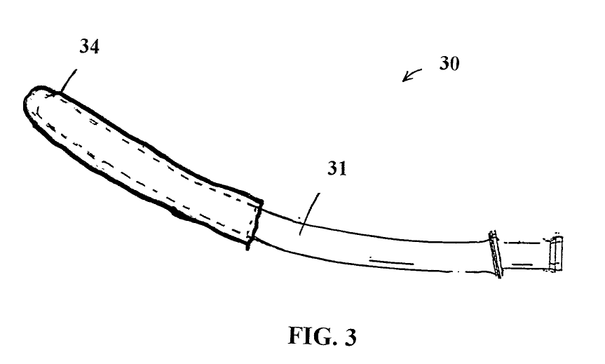

FIG. 3 shows an applicator 30 having a removable protective cap 34. Means for measuring pH of the vagina (not shown) may be attached to cap 34. Suitable means include indicating pH tape, but any of the known methods of measuring pH of vaginal secretions or of the vaginal wall may be used. Methods and means for measuring vaginal pH are described, for example, in U.S. Pat. No. 5,063,930 (Nucci) and U.S. Pat. No. 6,283,927 (Caillouette). The means may be alternately fastened or attached to the external surface of reservoir 31. Where the means for measuring pH, for example, pH paper, is attached to the removable cap, pH may be measured with the cap on, and then the cap may be removed for application of the cream to the vagina.

pH may be used for monitoring and documentation of symptoms, for example, in a nursing home situation. The procedure for such monitoring and documentation may include contacting the pH measuring means of a combination vaginal applicator/pH tester with the vagina and noting pH. Typically, pH is indicated by a color change of pH paper. If a high pH (4.5 or above) is noted, a dose of estrogen cream may be administered. A typical dose contains 0.5-1 g of an estrogen composition, but higher or lower doses may be used as desired. In some embodiments of the invention, estrogen is administered three times per week, in others, once per week, and in still others, as necessary. Treatment may be continued until a pH of 6 or less is measured. At that time, the estrogen dose may administered, and further treatment discontinued. Accordingly, vaginal pH may thereby be used to determine the duration of treatment.

In another aspect, the present invention relates to a kit for treatment of urogenital disorders associated with atrophic vaginitis in symptomatic women having vaginal pH of 4.5 or above. The kit includes a plurality of estrogen doses in the form of a gel, cream or ointment, a plurality of prefilled disposable vaginal applicators, and instructions for administering the estrogen doses intravaginally using the applicators. The kit may additionally include means for measuring vaginal pH, which may be attached to one or more applicators, as described above, or may be a separate instrument or device, or simply pH indicating tape.

In yet another aspect, the present invention relates to a combination device for measuring vaginal pH and for intravaginally administering a pharmaceutical composition in the form of a gel, cream or ointment. The combination device comprises:

an elongated curved tubular reservoir for containing the gel, cream or ointment, said reservoir having an outside diameter ranging from 0.25 to 0.375 inches;

a plunger, sealingly seatable within the reservoir and operable to displace the gel, cream or ointment therefrom; and means for measuring vaginal pH, disposed on an external surface of the applicator.

The means for measuring pH may attach to a removable protective cap, and the external surface of the removable protective cap, for contact with the vaginal wall, may lubricious.

The kits and combination devices of the present invention provide for a convenient, simple, easy to use method for diagnosing, documenting and treating urogenital disorders associated with atrophic vaginitis. Because of these advantages, compliance with treatment orders is generally improved, on the part of both the patient and the treatment provider, especially in the nursing home situation. In addition, documentation of symptoms by recording pH justifies the treatment, and especially the use of local estrogen.

1. A method for documenting, monitoring and treating urogenital disorders associated with atrophic vaginitis in symptomatic women having vaginal pH of 4.5 or above, said method comprising
a. measuring and recording vaginal pH; and
b. intravaginally administering at least one dose of an estrogen composition, in the form of a gel, cream or ointment, using a prefilled applicator comprising
i. a single dose of the estrogen composition;
ii. an elongated curved or angled tubular reservoir containing the single dose, said reservoir having an outside diameter ranging from 0.25 to 0.375 inches; and
iii. a plunger, sealingly seatable within the reservoir and operable to displace the estrogen composition therefrom.

2. A method according to claim 1, wherein the reservoir comprises a soft, flexible polymer composition.

3. A method according to claim 2, wherein the soft, flexible polymer composition is selected from high density polyethylene and medical grade PVC.

4. A method according to claim 1, wherein at least a portion of an external surface of the applicator is lubricious.

5. A method according to claim 1, wherein the applicator additionally comprises a removable protective cap having a lubricious external surface.

6. A method according to claim 1, wherein vaginal pH is used to determine the duration of treatment.

7. A kit for treatment of urogenital disorders associated with atrophic vaginitis in symptomatic women having vaginal pH of 4.5 or above, said kit comprising
a. a plurality of single doses of an estrogen composition, in the form of a gel, cream or ointment; and
b. a plurality of prefilled, disposable vaginal applicators, each applicator comprising:
i. an elongated curved or angled tubular reservoir containing a single dose of an estrogen composition, the average outside diameter of the reservoir ranging from about 0.25 to about 0.375 inches; and
ii. a plunger, sealingly seatable within the reservoir and operable to displace the gel, cream or ointment therefrom; and
c. instructions for administering the estrogen composition intravaginally using the applicator.

8. A kit according to claim 7, wherein the reservoir comprises a soft, flexible polymer composition.

9. A kit according to claim 8, wherein the soft, flexible polymer composition is selected from high density polyethylene and medical grade PVC.

10. A kit according to claim 7, additionally comprising means for measuring vaginal pH.

11. A kit according to claim 7, wherein at least a portion of an external surface of the applicator is lubricious.

12. A prefilled vaginal applicator for intravaginal administeration of an estrogen composition in the form of a gel, cream or ointment, the applicator comprising:
a. a single dose of the estrogen composition;
b. an elongated curved or angled tubular reservoir containing the estrogen composition, said reservoir having an outside diameter ranging from 0.25 to 0.375 inches; and
c. a plunger, sealingly seatable within the reservoir and operable to displace the estrogen composition therefrom.

13. A vaginal applicator according to claim 12, wherein the elongated tubular reservoir is curved.

14. A vaginal applicator according to claim 12, wherein the elongated tubular reservoir is angled.

15. A vaginal applicator according to claim 12, wherein the elongated tubular reservoir has a lubricious external surface for contact with a vaginal wall.

16. A combination device for measuring vaginal pH and for intravaginally administering a pharmaceutical composition in the form of a gel, cream or ointment, said combination device comprising:
a. an elongated curved or angled tubular reservoir for containing the gel, cream or ointment, said reservoir having an outside diameter ranging from 0.25 to 0.375 inches;
b. a plunger, sealingly seatable within the reservoir and operable to displace the gel, cream or ointment therefrom; and
c. means for measuring vaginal pH, disposed on an external surface of the applicator.

17. A combination device according to claim 16, wherein the means for measuring pH is attached to a removable protective cap.

18. A combination device according to claim 17, wherein at least a portion of an external surface of the removable protective cap, for contact with the vaginal wall, is lubricious.

19. A method for treating urogenital disorders associated with atrophic vaginitis in symptomatic women having vaginal pH of 4.5 or above, said method comprising intravaginally administering at least one dose of an estrogen composition, in the form of a gel, cream or ointment, using a prefilled applicator comprising
a. a single dose of the estrogen composition;
b. an elongated curved or angled tubular reservoir containing the single dose, said reservoir having an outside diameter ranging from 0.25 to 0.375 inches; and
c. a plunger, sealingly seatable within the reservoir and operable to displace the estrogen composition therefrom.

20. A method according to claim 19, wherein the reservoir comprises a soft, flexible polymer composition.

21. A method according to claim 20, wherein the soft, flexible polymer composition is selected from high density polyethylene and medical grade PVC.

22. A method according to claim 19, wherein at least a portion of an external surface of the applicator is lubricious.

23. A method according to claim 19, wherein the applicator additionally comprises means for measuring vaginal pH, disposed on an external surface of the applicator.
